(19) United States
(12) Patent Application Publication
(10) Pub. No.: US 2019/0307180 A1
(43) Pub. Date: Oct. 10, 2019

(54) BRASSIERE INCORPORATING A HOT FLASH MITIGATION SYSTEM

(71) Applicants: Margaret Githuku, Mason, OH (US); David Githuku, Mason, OH (US); Cory Mack, Venice, CA (US)

(72) Inventors: Margaret Githuku, Mason, OH (US); David Githuku, Mason, OH (US); Cory Mack, Venice, CA (US)

(21) Appl. No.: 16/379,905

(22) Filed: Apr. 10, 2019

Related U.S. Application Data
(60) Provisional application No. 62/655,399, filed on Apr. 10, 2018.

(51) Int. Cl. A41C 3/00; A61F 7/00; A61B 5/00

(52) U.S. Cl. CPC A41C 3/0064; A61F 7/00; A61B 5/0024; A61F 2007/0068; A61B 5/6804; A61F 2007/0011; A61F 2007/0021; A61B 5/4836

(57) ABSTRACT

A brassiere incorporating a hot flash mitigation system for cooling the upper torso, chest and neck area of a wearer is disclosed. The brassiere comprises two front panel sections connectively engaged to each other, at least one rear panel section connectively engaged to each of the two front panel sections. The hot flash mitigation system provides a deformable reservoir for containing and dispensing a fluid therefrom. The deformable reservoir is disposed in mating and contacting engagement with the brassiere and has an outlet in cooperative fluid engagement thereto. At least one duct disposed proximate to one of the at least two front panel sections is in fluid engagement with the outlet of the deformable reservoir. At least one nozzle disposed upon an end of the duct distal from the deformable reservoir operatively conveys the fluid from the duct to the upper torso, chest and neck area of the wearer.

### FIELD OF THE INVENTION

The present disclosure relates to a garment that supports the female mammary glands and provides cooling to the upper torso, chest and neck area of the wearer. The present disclosure more particularly relates to a garment suitable for assisting in the relief of the hot flashes associated with menopause and perimenopause by expelling air to targeted areas of the body such as the neck, chest, arms, back, or other regions of the body proximate to the brassiere.

### BACKGROUND OF THE INVENTION

Menopause is the time in a woman's life when the function of the ovaries ceases and she can no longer become pregnant. The ovary (female gonad), is one of a pair of reproductive glands in women. They are located in the pelvis, one on each side of the uterus. Each ovary is about the size and shape of an almond. The ovaries produce eggs (ova) and female hormones such as estrogen. During each monthly menstrual cycle, an egg is released from one ovary. The egg travels from the ovary through a Fallopian tube to the uterus.

The ovaries are the main source of female hormones, which control the development of female body characteristics such as the breasts, body shape, and body hair. The hormones also regulate the menstrual cycle and pregnancy.

Menopause is defined as the state of an absence of menstrual periods for 12 months. The menopausal transition starts with varying menstrual cycle length and ends with the final menstrual period. Perimenopause is a term sometimes used and means “the time around menopause.” It is often used to refer to the menopausal transitional period. Perimenopause is sometimes used to explain certain aspects of the menopause transition in lay terms.

Menopause occurs because of the sharp decrease of estradiol and progesterone production by the ovaries. After menopause, estrogen continues to be produced mostly by aromatase in fat tissues and is produced in small amounts in many other tissues such as ovaries, bone, blood vessels, and the brain where it acts locally. The substantial fall in circulating estradiol levels at menopause impacts many tissues, from brain to skin. Additionally, in contrast to the sudden fall in estradiol during menopause, the levels of total and free testosterone, as well as dehydroepiandrosterone sulfate (DHEAS) and androstenedione appear to decline more or less steadily with age. An effect of natural menopause on circulating androgen levels has not been observed.

Before menopause, a woman's periods typically become irregular, which means that periods may be longer or shorter in duration or be lighter or heavier in the amount of flow. During this time, women often experience hot flashes and other vasomotor symptoms. A hot flash is a feeling of warmth that spreads over the body and is often most pronounced in the head and chest. A hot flash is sometimes associated with flushing and is sometimes followed by perspiration. These menopausal transition symptoms typically last from 30 seconds to ten minutes and may be associated with shivering, sweating, and reddening of the skin. Although the exact cause of hot flashes is not fully understood, hot flashes are likely due to a combination of hormonal and biochemical fluctuations brought on by declining estrogen levels.

There is currently no method to predict when hot flashes will begin and how long they will last. Hot flashes occur in up to 40% of regularly menstruating women in their forties, so they may begin before the menstrual irregularities characteristic of menopause even begin. About 80% of women will be finished having hot flashes after five years. Sometimes (in about 10% of women), hot flashes can last as long as 10 years. There is no way to predict when hot flashes will cease, though they tend to decrease in frequency over time. They may also wax and wane in their severity. The average woman who has hot flashes will have them for about five years.

The current management of hot flashes generally consist of managing the current lifestyle. Current hot flash mitigation management techniques only include measures such as drinking cold liquids, staying in cool rooms, using fans, removing excess clothing, and avoiding hot flash triggers such as hot drinks, spicy foods, etc. These techniques may even be partially supplemented by the use of medication.

A brassiere (or bra) is a form-fitting undergarment designed to support a woman's breasts. Mass-produced bras are manufactured to fit a prototypical woman standing with both arms at her sides.

A bra is typically a complicated garment to make. A common design can have between 20 and 48 component parts, including the band, hooks, cups, lining, and straps. The main components of a brassiere provide for a chest band that wraps around the torso, two cups attached thereto, and accompanying shoulder straps. The chest band is usually closable in the back by the use of a hook and eye fastener. However, the chest band may also be fastened at the front. Sleep bras or athletic bras do not have fasteners and are pulled on over the head and breasts. The section between the cups is called a gore. The section under the armpit where the band joins the cups is called the “back wing”.

Bra components, including the cup top and bottom (if seamed), the central, side and back panels, and straps, are cut to manufacturer's specifications. Many layers of fabric may be cut at the same time using computer-controlled lasers or bandsaw shearing devices. The pieces are assembled by piece workers using industrial sewing machines or automated machines. Coated metal hooks and eyes are sewn in by machine and heat processed or ironed into the back ends of the band and a tag or label is attached or printed onto the bra itself.

The chest band and cups of a typical brassiere, not the shoulder straps, are designed to support the weight of women's breasts. Strapless bras rely on an underwire and additional seaming and stiffening panels to support them. The shoulder straps of some sports bras cross over at the back to take the pressure off the shoulders when arms are raised. In short, a brassiere is intended to physically maintain the shape of the bust in a manner that intends to maintain, and not alter, the original or biologically presented bust or breast material.

As shown in FIGS. 1 and 2, an exemplary prior art brassiere (bra) 100 (greatly simplified from the construction detailed above solely for reasons of brevity) can generally comprise two mirror-image front panel sections 120 each in the general form of a cup (and known to those of skill in the art as a cup(s)) and at least two rear panel sections 110 (known to those of skill in the art as strap(s)). In some embodiments the at least two rear panel sections 110 can be cooperatively attached in mating engagement with each other with a clip, fastener, hook and loops, and the like so that the brassiere 100 may be easily placed (by placing the ends of each rear panel section in connective engagement) and/or removed (by disassociating the ends of each rear panel section from connective engagement). In other embodiments the two rear panel sections 110 may be cooperatively attached via a sewn seam or other form of fixable attachment known to those of skill in the art. In other embodiments, a single rear panel made of elastic material(s) can extend from a portion of one front panel section 120 to a portion of the other front panel section 120 and is meant to be worn across the back of the wearer.

As shown in the exemplary front elevational view of FIG. 2, the two front panels 120 may be connected via a connecting member 130. This member may be a seam, elastic material, a strap, or simply the region that connects the left and right 120 two front panels 120.

As shown in FIGS. 1 and 2, exemplary embodiments of the prior art may incorporate a respective shoulder strap 140 that extends from a first position disposed upon a respective front panel 120 over the shoulder of the wearer and cooperatively interfacing with a respective rear panel section 110 cooperatively associated with the respective front panel 120. The shoulder strap may interface with the respective front panel 120 and respective rear panel section 110 via a stitch or seam or via a fastener such as a grommet or loop. For increased comfort of the wearer, the shoulder strap 140 may be adjustable in length, toughness, or angle.

One of skill in the art will readily appreciate that there are no currently known devices that can be incorporated or are presently incorporatable with existing brassiere technology that would be useful to mitigate the symptoms associated with a menopausal hot flash. Therefore, there is a need for, and a clear victory for, a brassiere that could provide at least partial relief from menopausal hot flashes by the use of a motive fluid (e.g., air) and/or an invasive fluid (e.g., air). Additionally, there is a clear need for a menopausal hot flash mitigation system for incorporation into existing brassiere constructions and technology to provide at least partial relief from menopausal hot flashes.

### SUMMARY OF THE INVENTION

The present disclosure provides for a brassiere incorporating a hot flash mitigation system for cooling the upper torso, chest and neck area of a wearer. The brassiere incorporating a hot flash mitigation system provides a brassiere having two front panel sections connectively engaged to each other and at least one rear panel section connectively engaged to each of the two front panel sections. The connectively engaged two front panel sections and the at least one rear panel section forming a wrap capable of encircling the wearer or intended wearer's body. The hot flash mitigation system provides for a deformable reservoir for containing and dispensing a fluid therefrom. The deformable reservoir is disposed in mating and contacting engagement with the brassiere. The deformable reservoir has an outlet in cooperative fluid engagement thereto. The brassiere also provides for at least one duct disposed proximate to one of the at least two front panel sections and in fluid engagement with the outlet of the deformable reservoir. The brassiere further provides for at least one nozzle disposed upon an end of the duct distal from the deformable reservoir and in fluid communication with the at least one duct, the at least one nozzle operatively conveying the fluid from the duct to the upper torso, chest and neck area of the wearer.

The present disclosure also provides for a garment formed from a material. The garment provides for a deformable reservoir for containing and dispensing a fluid therefrom. The deformable reservoir is disposed in mating and contacting engagement with the garment. The deformable reservoir comprising an outlet in cooperative fluid engagement thereto. The garment also provides for at least one duct disposed proximate to a surface of the garment and in fluid engagement with the outlet of the deformable reservoir. The garment further provides for at least one nozzle disposed upon an end of the duct distal from the deformable reservoir and in fluid communication with the at least one duct. The at least one nozzle operatively conveys the fluid from the duct to the upper torso, chest and neck area of the wearer.

### BRIEF DESCRIPTION OF THE DRAWINGS

FIG. 1 is a front ¾ elevational view of an exemplary prior art brassiere;

FIG. 2 is a front elevational view of the exemplary prior art brassiere of FIG. 1;

FIG. 3 is a front ¾ elevational view of an exemplary brassiere incorporating the hot flash mitigation system of the present disclosure;

FIG. 4 is a front elevational view of the exemplary brassiere incorporating the hot flash mitigation system of FIG. 3;

FIG. 5 is a front ¾ elevational view of an exemplary brassiere incorporating the hot flash mitigation system of the present disclosure further incorporating at least one rear panel section with cooperatively associated ducts;

FIG. 6 is a front elevational view of an exemplary brassiere incorporating the hot flash mitigation system of FIG. 3 where certain ducts within the brassiere provide hot flash mitigation to the torso of the wearer;

FIG. 7 is a front ¾ elevational view of an exemplary garment incorporating the hot flash mitigation system of the present disclosure; and,

FIG. 8 is a front elevational view of an exemplary brassiere incorporating the hot flash mitigation system and a wireless body sensor network.

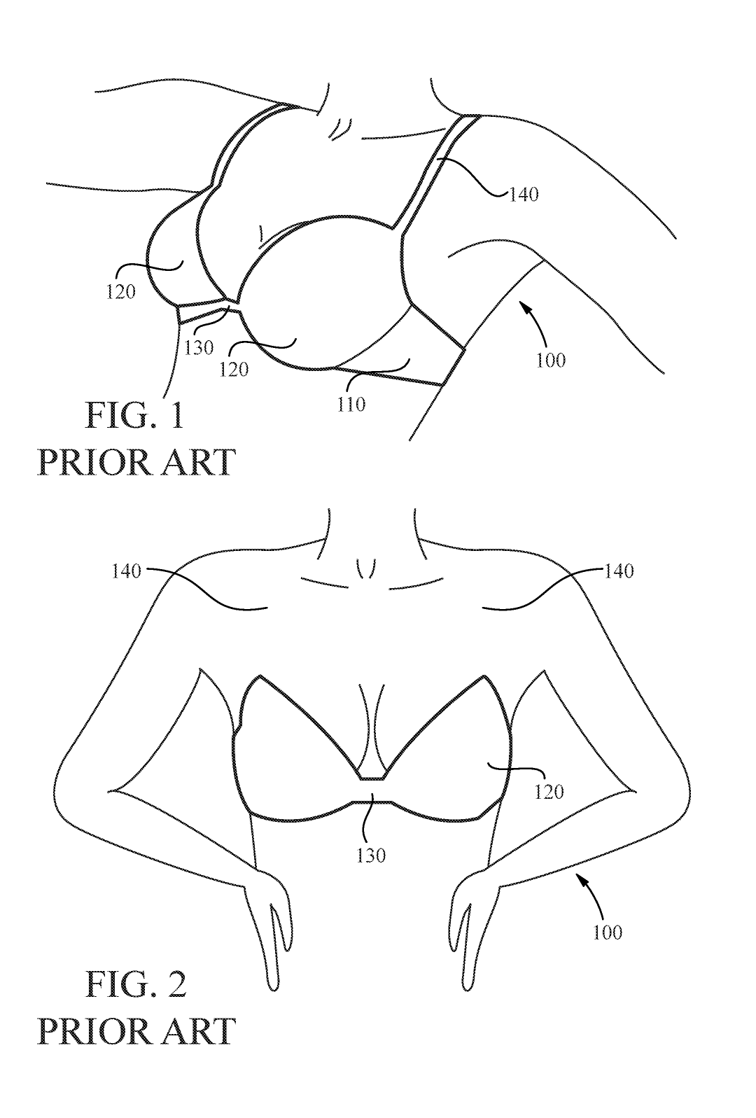

### DETAILED DESCRIPTION OF THE INVENTION

As used herein, a “fluid” is a substance that continually deforms (flows) under an applied shear stress. Fluids are a subset of the phases of matter and include liquids, gases, plasmas, and to some extent, plastic solids and flowable solids. A typical fluid suitable for use with the present disclosure is air. When used herein, the term “air” is merely illustrative and is intended to be used only as a non-limiting example of a fluid. In other words, “air” is intended to be an exemplary and non-limiting embodiment of a “fluid”. One of skill in the art would also understand that a fluid suitable for use with the present disclosure could also be considered a motive fluid (e.g., air) and/or an invasive fluid (e.g., air). One of skill in the art will also understand that a fluid may also have functional additives provided therein. Such functional additives could include perfumes, medicaments, therapeutic agents, combinations thereof and the like.

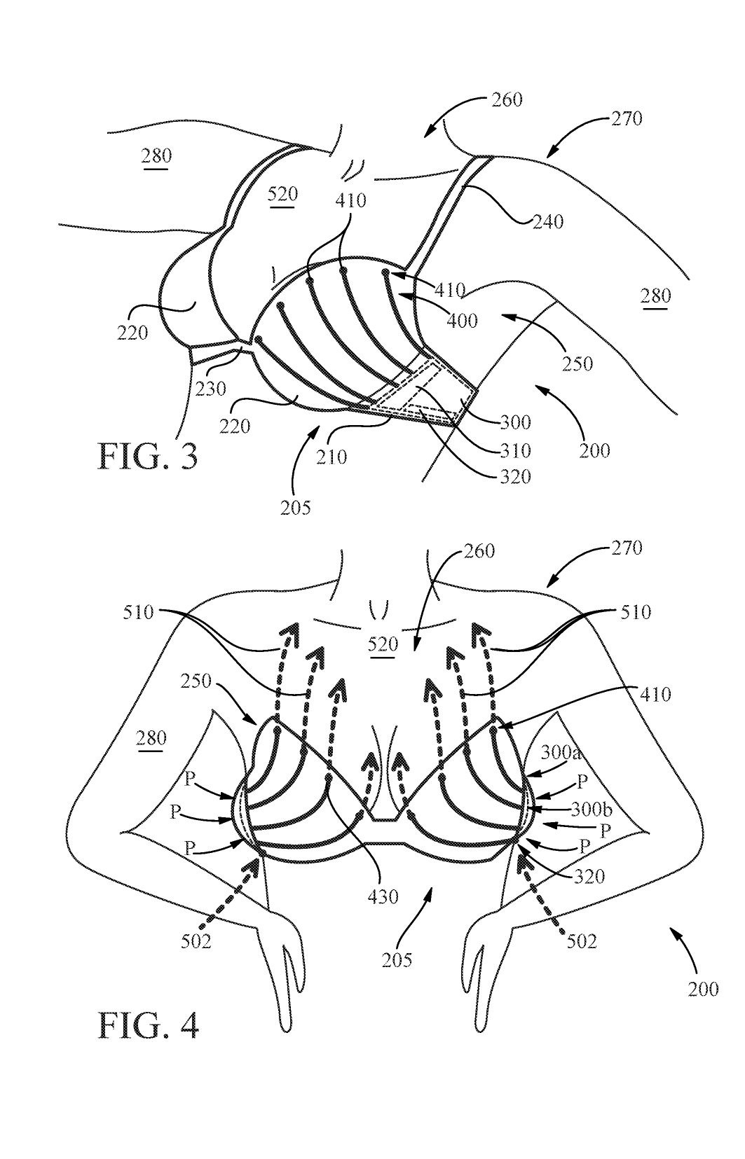

FIGS. 3 and 4 of the present disclosure depict an exemplary and non-limiting brassiere 200 incorporating the hot flash mitigation system 205. As indicated supra, a brassiere is typically an assemblage of a plurality of parts. However, for the sake of brevity, the brassiere 200 of the present disclosure will be generally assembled from a few individual components.

One of skill in the art will recognize that the exact construction of the brassiere 200 is not a critical element of the present disclosure. Thus, the construction of the brassiere 200 can follow any design norm useful for providing a brassiere 200. In other words, these generally described individual components may in reality be a complex assembly of a plurality of components as indicated supra. On the other hand, for purposes of simplicity, the components of a brassiere 200 may be comprised of a few parts derived and/or designed as combinations or sub-combinations of the aforementioned brassiere components. In any regard the brassiere 200 of the present disclosure may be considered, constructed, and described in any manner consistent with either an assembly of a plurality of completely integrated components, a simpler assembly of components (described infra), or a purposeful combination of components required to produce a brassiere as would be understood by one of skill in the art. The process and complexity of brassiere construction should not detract from the present description.

Generally, a brassiere 200 of the present disclosure generally comprises two front panel sections 200 (also known to those of skill in the art as cups 200), at least one rear panel section 210 (also known to those of skill in the art as a strap 210), and optional shoulder straps 240. A top hole 260 provides for placement of the brassiere 200 over the head and over the shoulders 270 of the wearer. A pair of opposed arm holes 250 each disposed upon opposed sides of the brassiere 200 can provide for the insertion of the arms 280 therethrough. Here, portions of fabric disposed between the top hole 260 and each of the arm holes 250 can capably form the optional shoulder straps 240 that can properly overlay the wearer's shoulders 270. However, referring to FIG. 4, one of skill in the art will recognize that shoulder straps 240 are not completely necessary. In some constructions, it may be desirable that the two front panel sections 200 can only be connectively engaged to the at least one rear panel section 210 (also referred to herein as back panel 225) to form a de facto wrap that encircles the wearer or intended wearer's body without the need for shoulder straps 240.

The two front panels 220 may be fixably and permanently connected via a connecting member 230. This member may be a seam, an elastic material, a strap, or simply a region of connection between the mirror image left and right two front panels 220. Alternatively, the two front panels 220 may be releasably connected to each other at opposing edges for securing the two front panels 220 together when the brassiere 200 is worn.

Additionally, the at least one rear panel section 210 can be provided as a plurality of connectable segments. In a preferred embodiment, a first of the connectable segments (or sections) is fixably attached to one of the two front panels 220 and a second connectable section being fixably attached to the other of the two front panel sections 220. Each of the plurality of connectable sections can be cooperatively attached in mating engagement to each other at an end distal from the fixable connection to the respective panel section of the two front panels 220 with a clip, fastener, hook and loops, and the like, so that the brassiere 200 may be easily placed (by placing the ends of each rear panel section in connective engagement) and/or removed (by disassociating the ends of each rear panel section from connective engagement). In some embodiments, two rear panel sections 210 are attached with a clip or fastener so that it may be easily removed. In other embodiments, two rear sections 210 may be attached via a seam, and in others still there is one rear panel section 210 made of an elastic material that can extends from one front panel section 220 to the other front panel section 220 and is meant to be worn across the back of the wearer. In other embodiments, two rear panel sections forming the at least one rear panel section 210 may be cooperatively attached via a sewn seam or other form of fixable attachment known to those of skill in the art. In other embodiments, a single rear panel section 210 made of an elastic material(s) can extend from a portion of one front panel section 220 to a portion of the other front panel section 220 (forming a loop) and is meant to be worn across the back of the wearer. It is believed that other methods of facilitating the securement of the brassiere 200 to the wearer are feasible for use and can be successfully incorporated with the brassiere 200 envisioned for use herein.

As mentioned supra, a shoulder strap 240 can extend from a respective panel section of the two front panel sections 220 over the wearer's shoulders 270 and interfacing with the at least one rear panel section 210. The shoulder strap may interface and connectively engage with a respective panel section of the two front panel sections 220 and the at least one rear panel section 210 via a stitch, seam, or fastener such as a grommet or loop. For increased comfort of the wearer, the shoulder strap 240 may be adjustable in length, toughness, or angle.

As shown in FIGS. 3 and 4, a hot flash mitigation system 205 incorporating a deformable reservoir 300 can be disposed in mating and contacting engagement with brassiere 200. In a preferred embodiment, a deformable reservoir 300 can be disposed proximate to each respective arm hole 250 and proximate to the area of connecting engagement between a front panel section 200 and a corresponding rear panel section 210 (i.e., the back wing). In function, the deformable reservoir 300 is capable of containing and dispensing a fluid therefrom. The deformable reservoir 300 has a fluid chamber containing the fluid to be dispensed. The deformable reservoir 300 can be constructed as a re-inflatable bag or pouch. The fluid chamber or a wall bounding the fluid chamber (in this case the deformable reservoir 300 wall) is preferably of a flexible, deformable, collapsible, and/or repeatedly collapsible construction, at least in parts. By way of non-limiting example, the deformable reservoir 300 may constructed of an open cell foam that allows fluid (for example ambient air) repeatedly and sequentially enter deformable reservoir 320 via an inlet 320 and exit via an outlet 310.

The design of a preferred embodiment of the inlet 320 may be that of a one-way valve 320 (or check valve 320), or similar device, that would allow a fluid to enter but not exit the deformable reservoir 300. Such a one-way valve 320 can be heat welded or even glued onto the material forming deformable reservoir 300 or otherwise incorporated into the materials forming deformable reservoir 300. For example, a one-way valve 320 can be formed from an inner film and a barrier layer that are both perforated, preferably by means of cuts, pricks, or stampings, to form a flow opening. The area under the flow opening is bonded to a film that is not laminated to the barrier layer, forming a longitudinal channel. The longitudinal channel can extend the entire width of the laminate so that during manufacture, the channel extends to the material forming deformable reservoir 300. Such a construction would allow a fluid to flow into deformable reservoir 300 when the interior portion of deformable reservoir 300 is provided with a negative pressure gradient and prevent the outflow of fluid within the deformable reservoir 300.

The outlet 310 of deformable reservoir 300 may be that of an opening, slit, or one-way valve similar to, or as described relative to, one-way valve 320 supra. In some embodiments outlet 310 may be a region that acts as plenum 310 and may also have the function of one-way valve in that the fluid disposed and/or contained within deformable reservoir 300 can exit deformable reservoir 300 through outlet 310 when an external pressure is applied to the surface of deformable reservoir 300. Upon the egress of fluid from the deformable reservoir 300 through outlet 310, a negative pressure relative to the atmosphere exists internal to deformable reservoir 300 thereby facilitating the entry of new fluid (e.g., air) into the deformable reservoir 300 via one-way valve 320. In this manner, deformable reservoir can function as a re-inflatable balloon (or as a lung) by sequentially taking in fluid, expelling fluid, and taking in additional, new fluid).

As shown in FIGS. 3 and 4, the hot flash mitigation system 205 can provide for at least one or more ducts 400 may each be fluidly connected to (i.e., in fluid engagement with) the outlet 310. By way of non-limiting example, a plurality of ducts 400 are each connected to outlet 310. Each duct 400 of the hot flash mitigation system 205 can be provided with a nozzle 410. It is believed that the presence of multiple ducts 400 can provide increased comfort for the relief of a hot flash as the fluid (e.g. air) conveyed from outlet 310 to nozzle 410 through duct 400 can be directed to multiple parts of the body such as upper torso, chest and neck area 520 of the wearer (this flow is shown by the arrows 510) as desired.

Without desiring to be bound by theory, it is believed that one of skill in the art would understand that the increased comfort provided by the brassiere 200 from the fluid (e.g. air) expelled from the nozzle 410 toward the upper torso, chest and neck area 520 of the wearer can be attributable to the Joule-Thomson effect. In thermodynamics, the Joule-Thomson effect describes the temperature change of a real gas or liquid when it is forced through a valve while keeping the valve insulated so that no heat is exchanged with the environment. At room temperature, a fluid (e.g., air) expelled from a nozzle cools upon adiabatic expansion by the Joule-Thomson process.

In practice, the Joule-Thomson effect is achieved by allowing a gas to expand through a throttling device (such as the nozzle 410 described supra). No external work is extracted from the gas during this expansion. The cooling produced from this expansion is suitable for use in cooling processes.

As would be understood by one of skill in the art, two factors can change the temperature of a fluid during adiabatic expansion: 1. a change in internal energy of the fluid or 2. the conversion of the fluid's potential energy to kinetic internal energy. It is understood that temperature is the measure of thermal kinetic energy (i.e., the energy associated with molecular motion). Thus, a change in temperature of a fluid indicates a change in thermal kinetic energy. Since the internal energy of the fluid is the sum of the thermal kinetic energy and the thermal potential energy, even if the internal energy does not change, the temperature can change due to conversion between kinetic and potential energy. In short, an adiabatic free expansion typically produces a decrease in temperature of the fluid as it expands in volume. This expansion can provide a large cooling effect from a fluid escaping through a nozzle such as nozzle 410 of the present disclosure.

In function, the brassiere 200 of the present disclosure can provide mitigating and therapeutic relief prior to, at the outset of, and during a hot flash. The reservoir 300 intakes a fluid (e.g., air) by providing a region of negative pressure relative to the atmosphere that facilitates the ingress of the fluid though the one-way valve 320. At the onset of a hot flash, or during a hot flash, the wearer can maneuver the arms toward reservoir 300 (e.g., in the direction of arrows labelled ‘P’) and provide a positive pressure to the outside of reservoir 300 to create a positive pressure relative to the atmosphere that exhausts the fluid contained within the reservoir 300 outward through outlet 310 (preferably provided as a plenum) into each duct 400 operatively and fluid communicatively connected to outlet 310 toward nozzle 410 and outward therefrom to the upper torso, chest and neck area 520 of the wearer. As pressure is released by the wearer's arms from the surface of reservoir 300, reservoir 300 is then provided with a negative internal pressure relative to the ambient environment facilitating the ingress of new fluid (e.g., air) into the reservoir through one-way valve 310. This easily facilitates the multiple and repeatable action of allowing the wearer to actuate the hot flash mitigation system 205 and provide a hot flash mitigating air flow to the upper torso, chest and neck area 520 of the wearer by repeated pressing (i.e., pressurizing) of the reservoir 300 operable connected to the brassiere 200. In other words, the reservoir 300 operates in a balloon-like manner to repeatably and consistently intake air from the surrounding environment and expel air to the upper torso, chest and neck area 520 of the wearer. In short, the reservoir 300 operates in a lung-like manner. This is a completely different process than what one of skill in the art would consider a ‘closed-loop’ system such as a refrigeration loop. One of skill in the art would understand that a closed-loop system employs a pumped fluid that circulates in a closed loop without any exposure to the local environment and typically without the transfer of fluid into or out of the closed loop. One of skill in the art would understand that the present device provides an ‘open-loop’ system. One of skill in the art would understand that an open-loop system provides for the pumped fluid to be exposed to the local atmosphere at some point in the circuit.

Further, one of skill in the art could provide for a fluid that may also have functional additives provided therein. Such functional additives could include perfumes, medicaments, therapeutic agents, combinations thereof and the like. Further, one of skill in the art will readily recognize that function additives such as perfumes, medicaments, therapeutic agents, combinations thereof and the like could be directly added to, or provided in cooperation with, the reservoir 300. In other words, a functional additive such as a medicament could be directly added to the internal portion of reservoir 300 by incorporating the functional additive with the fluid taken in by reservoir 300 during routine use. Alternatively, a functional additive such as a medicament would be input directly into the interior of reservoir 300. Yet still, a functional additive such as a perfume could be incorporated into the materials used to form reservoir 300. All of these embodiments should be considered exemplary and non-limiting.

The ducts 400 may be constructed of any material that allows the fluid to be conveyed to the upper torso, chest and neck area 520 of the wearer. This includes but is not limited to plastic or metals. In a preferred embodiment the ducts 400 may be constructed of a soft flexible silicone rubber. The ducts 400 nay have a narrow cross-section. This may be done to make the construction, function, and purpose of the brassiere 200 less obvious to passers-by, resulting in increased emotional comfort of the wearer. Each duct may be directly attached to the material forming each of the two front panel sections 220, interwoven into the material forming each of the two front panel sections 220, or even comprise the material forming each of the two front panel sections 220.

For example, the material used to form a front panel section 220 can comprise discrete hollow fibers (such as capillary fibers). The discrete hollow fibers can be arranged in such a manner that they are provided with a cross sectional size and provided in a distribution that can facilitate the fastest cooling of the upper torso, chest and neck area 520 by facilitating the fastest movement of fluid from the reservoir 300. An exemplary but non-limiting distribution of discrete hollow fibers that can enable an efficient flow of fluid from the reservoir 300 to the upper torso, chest and neck area 520 could be in a fractal pattern similar to natural patterns found in root systems, capillary vessels, and the alveoli in lungs.

Such discrete hollow fibers of the hot flash mitigation system 205 can be interwoven into the material forming the two front panel sections 220. This construction can facilitate the connection of each fiber to reservoir 300. It is also possible that each duct 400 be perforated to allow fluid (e.g., air) to escape along the path of the duct 400 to dispense the fluid contained therein at multiple points onto parts of the chest along the path of each duct 400 disposed within or upon the material forming the two front panel sections 220.

Further, as shown in FIG. 5 the material used to provide the two front panel sections 220 as well as the material used to provide the at least one rear panel section 210 can be provided with cooperatively associated ducts 400, 400B respectively. Here, ducts 400, 400B can direct the flow of fluid outward from each respective nozzle 410 toward the upper torso, chest and neck area 520 of the wearer (i.e., via ducts 400) as well as the upper back and neck 530 of the wearer (i.e., via ducts 400B). In this way, one of skill in the art will recognize that the brassiere 200 and the hot flash mitigation system 205 can cool two regions of the upper torso (i.e., the upper torso, chest and neck area 520 and the upper back and neck 530) of the wearer with a single garment when the user experiences a hot flash.

As mentioned supra, a nozzle 410 may be attached to each duct 400 at an end distal from reservoir 300. The nozzles 410 may be used to adjust the flow of air for more air flow or it may be used to restrict airflow, so the fluid doesn't move the wearer's hair.

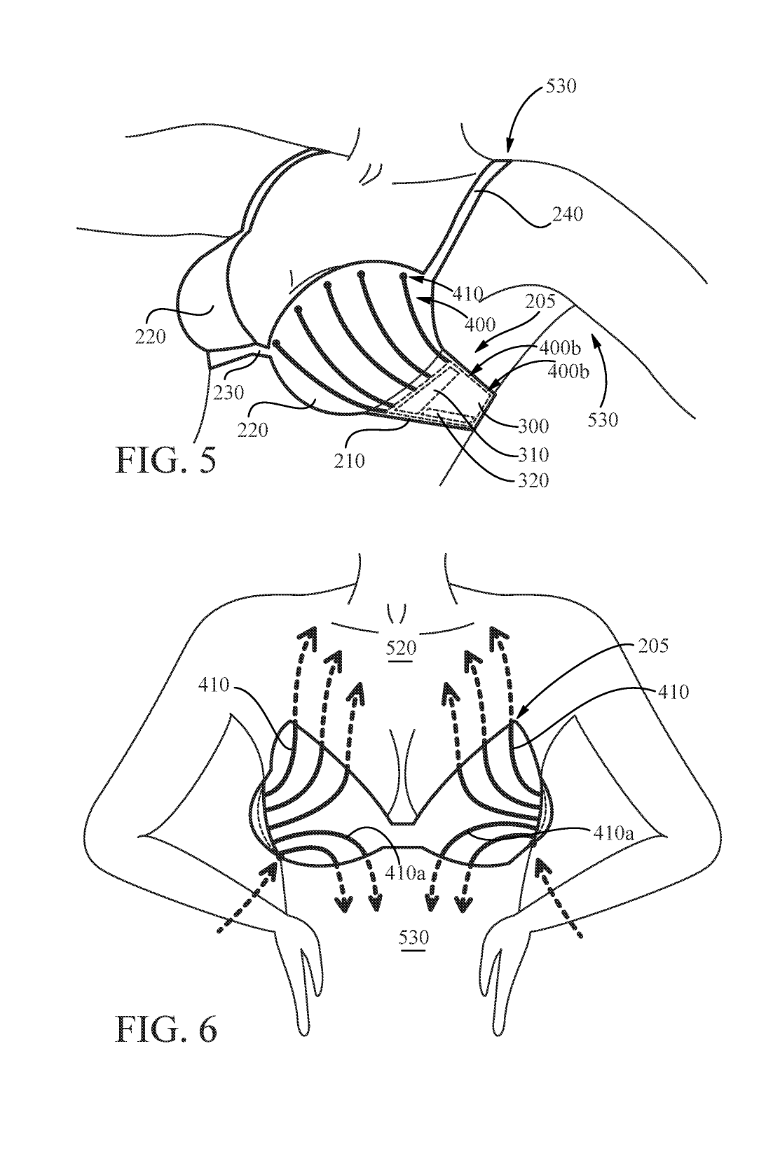

As shown in FIG. 6, ducts 410A and respective nozzles 410 can be provided within the front panel 220 to direct the flow of fluid outward from each respective nozzle 410 toward the central and/or lower torso 520 (i.e., the region below the breast area) of the wearer. In a similar vein, one of skill in the art could provide both ducts 410 and ducts 410A within or within the respective front panel 220 to provide fluid flow toward both the central and/or lower torso 520 and the upper torso, chest and neck area 520 of the wearer.

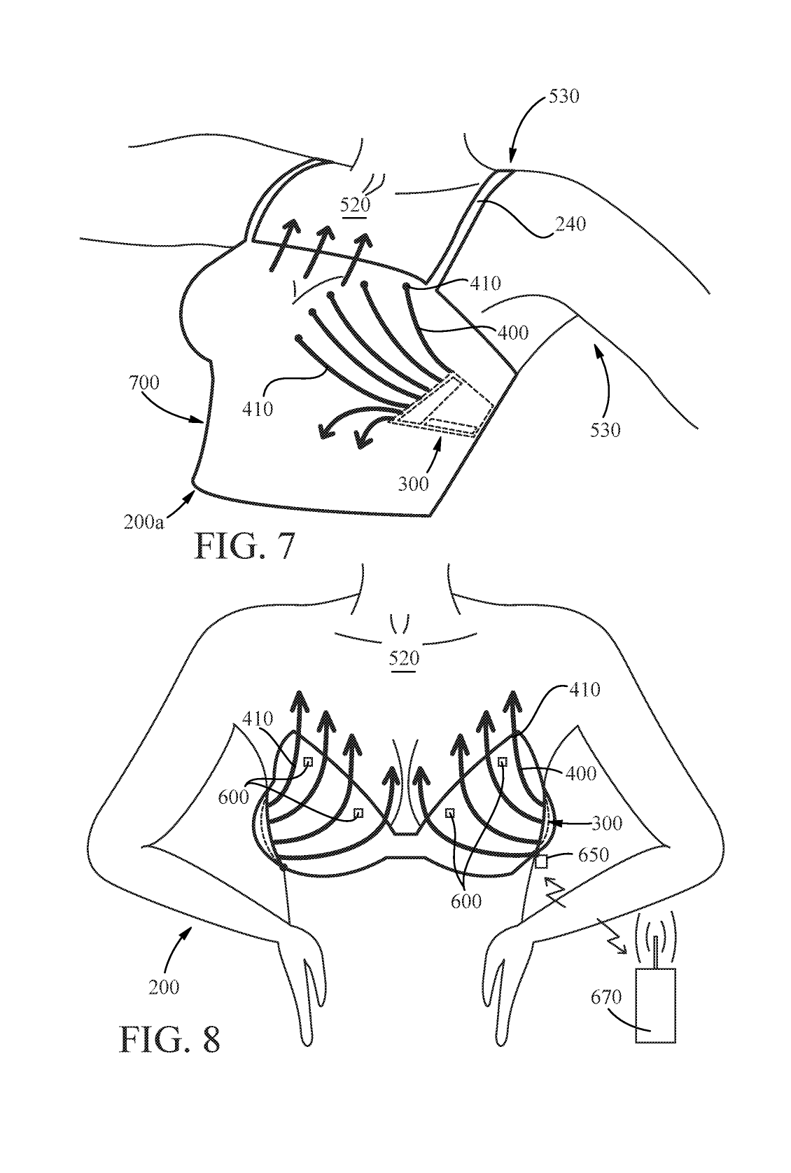

Additionally, as shown in FIG. 7, it should be readily recognized by one of skill in the art that the aforementioned structures (collectively or in part) could easily be incorporated into, and upon the surface of garments 200A other than brassieres 200. By way of non-limiting examples, the reservoir 300 (and the additional structures fluidly and cooperatively associated thereto) as well as the ducts 410 (and any additional structures fluidly and cooperatively associated thereto) could be incorporated into, or upon the surface of, wearable garments such as hats, socks, underwear, shirts, pants, and the like. For example, FIG. 7 provides an exemplary shirt 700 used for wearer workouts that could be provided with any number of reservoirs 30, ducts 400, and nozzles 410 that could facilitate the cooling of the individual as the wearer's work out progresses, the body's core temperature rises, and sweat develops. Or such a shirt could be utilized by a hiker while hiking on a hot summer day to lower the body's core temperature by exhausting air toward regions of the body having major blood vessels.

As shown in FIG. 8, the brassiere 200 and hot flash mitigation system 205 can incorporate a wireless body sensor network (or simply BSN) 600. A BSN 600 is a networked collection of a plurality of (programmable) sensor nodes that can communicate among themselves, with other smart devices 670, and other ambient sensors. The sensor nodes can have computation, storage, wireless transmission, and sensing capabilities. Common physiologically sensed signals/data can include body motion, skin temperature, heart rate, skin conductivity, brain and muscle activity, and biomarkers. In a preferred embodiment, the BSN 600 is cooperatively associated with the physiological symptoms associated with menopause and/or hot flashes. To perform both online and offline analyses of data streams, BSNs 600 can be assisted by cloud computing-based infrastructures providing flexible storage and scalable processing. A wide range of application scenarios is enabled by BSN 600 technologies, even though m-Health applications probably represent the most emblematic and diffused example. Specifically, BSN 600-based systems can be used to directly monitor several vital signs (such as those associated with the aforementioned hot flashes) continuously and non-invasively, as tiny wireless BSN sensors 600 can be placed on material forming the brassiere 200 and in contacting engagement with the skin of the wearer. In other words, the BSN 600 can be integrated with garments 200A or in the materials and/or the fibers forming materials useful for garments 200A such as brassiere 200.

Moreover, BSNs can be strategic enablers for many other application domains such as: eSport, e-Fitness, e-Wellness, and e-Social. Existing BSN 600 research by one of skill in the art can craft an intimately integratable garment assembly (such as for brassiere 200) from several points of view: hardware (e.g., biosensor boards), communications (e.g. efficient MAC-level protocols), distributed software systems (e.g., collaborative smartphone- and/or BSN-based platforms), and novel applications including advanced data processing algorithms.

Additionally, BSNs 600 can be connected to cloud computing by embracing the concept of providing computer resources as a third-party service. Resources include storage, networking, and processing. Different cloud implementations offer different variations of available services. Realized benefits can include dynamic access to resources based on usage demands, and third party management of computing resources. Utilization of cloud computing resources can be provided at a platform level and utilized by customers (e.g., a FITbit). While attractive due to management and utilization efficiencies, there can be costs associated with cloud platforms or configuring BSN 600 data to work with clouds. Complexity may range from recompiling an application for a specific platform to substantial code modifications to access and utilize cloud APIs. Applications can run within closed or secure networks, or connected to identifiable and secure hardware, may operate without securing each individual communication or data transaction. Cloud access is generally over the Internet, rather than restricted to internal access, and hardware resources and connections may be fully under third party control.

In any regard to the integration of such BSN 600 with a garment such as brassiere 200 and/or the hot flash mitigation system 205, the onset of a hot flash can be readily detected by a BSN 600 (perhaps even prior to the wearer's sensation of the onset of a hot flash) and notify the wearer of the impending event via a communication link to a smart device 670 such as a smart phone. Alternatively, one of skill in the art could integrate a BSN 600 with an electromechanical device 650 that initiates the process of ejecting fluid (e.g., air) from the reservoir 300 cooperatively associated with the brassiere 200 through the ducts 400 in fluid communicating engagement thereto through each nozzle 410 cooperatively associated thereto and onto the upper torso, chest and neck area 520 of the wearer.

Any dimensions and/or values disclosed herein are not to be understood as being strictly limited to the exact numerical values recited. Instead, unless otherwise specified, each such dimension and/or value is intended to mean both the recited dimension and/or value and a functionally equivalent range surrounding that dimension and/or value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any 7 other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A brassiere 200 incorporating a hot flash mitigation system for cooling the upper torso, chest and neck area 520 of a wearer, said brassiere 200 incorporating a hot flash mitigation system comprising:
a) a brassiere comprising two front panel sections 220 connectively engaged to each other and at least one rear panel section 210 connectively engaged to each of said two front panel sections 220, said connectively engaged two front panel sections and said at least one rear panel section 210 forming a wrap capable of encircling the wearer or intended wearer's body; and,
b) a hot flash mitigation system 205 comprising:
1) a deformable reservoir 300 for containing and dispensing a fluid therefrom, said deformable reservoir 300 being disposed in mating and contacting engagement with said brassiere 200, said deformable reservoir comprising an outlet 310 in cooperative fluid engagement thereto;
2) at least one duct 400 disposed proximate to one of said at least two front panel sections 220 and in fluid engagement with said outlet 310 of said deformable reservoir 300; and,
3) at least one nozzle 410 disposed upon an end of said duct 400 distal from said deformable reservoir 300 and in fluid communication with said at least one duct 400, said at least one nozzle 410 operatively conveying said fluid from said duct to said upper torso, chest and neck area 520 of said wearer.

2. The brassiere 200 of claim 1 wherein said two front panel sections 220 and said at least one rear panel section 210 connectively engaged thereto collectively form a top hole 260, said top hole 260 providing for placement of said brassiere 200 over the head and shoulders 270 of said wearer.

3. The brassiere 200 of claim 1 further comprising at least one shoulder strap 240, said should strap being connectively engaged to one of said two front panel sections 220 and said at least one rear panel section 210 and an arm hole 250 for the insertion of arms 280 of the wearer therethrough.

4. The brassiere 200 of claim 1 wherein said deformable reservoir 300 further comprises a reinflatable bag or pouch.

5. The brassiere 200 of claim 4 wherein said deformable reservoir 300 is constructed from an open cell foam.

6. The brassiere 200 of claim 4 wherein said deformable reservoir 300 further comprises an inlet 320, said inlet 320 allowing fluid to enter said deformable reservoir 300 therethrough.

7. The brassiere 200 of claim 6 wherein said inlet 320 further comprises a one-way valve, said one-way valve allowing said fluid to enter and not exit said deformable reservoir 300.

8. The brassiere 200 of claim 4 wherein said outlet 310 of said deformable reservoir 300 comprises a one-way valve, said one-way valve allowing said fluid to exit and not enter said deformable reservoir 300.

9. The brassiere 200 of claim 1 wherein said one of said two front panel sections 220 is formed from a material, said duct 400 being contactingly and matingly engaged to said material.

10. The brassiere 200 of claim 1 wherein said one of said two front panel sections 220 is formed from a material, said duct 400 being incorporated within said material.

11. The brassiere 200 of claim 1 wherein said duct 400 is a discrete hollow fiber.

12. The brassiere 200 of claim 11 wherein said discrete hollow fiber is disposed within said material forming said one of said two front panel sections 220.

13. The brassiere 200 of claim 1 wherein said application of a pressure to said reservoir operatively conveys said fluid from said deformable reservoir 300, through said outlet 310 and into said duct 400, through said nozzle 410 and onto said upper torso, chest and neck area 520 of said wearer.

14. The brassiere 200 of claim 13 wherein said wearer applies said pressure to said reservoir upon the on-set of a hot flash.

15. A garment 200A formed from a material, said garment 200A comprising:
a) a deformable reservoir 300 for containing and dispensing a fluid therefrom, said deformable reservoir 300 being disposed in mating and contacting engagement with said garment 200A, said deformable reservoir comprising an outlet 310 in cooperative fluid engagement thereto;
b) at least one duct 400 disposed proximate to a surface of said garment 200A and in fluid engagement with said outlet 310 of said deformable reservoir 300; and,
c) at least one nozzle 410 disposed upon an end of said duct 400 distal from said deformable reservoir 300 and in fluid communication with said at least one duct 400, said at least one nozzle 410 operatively conveying said fluid from said duct to said upper torso, chest and neck area 520 of said wearer.

16. The garment 200A of claim 15 wherein said deformable reservoir 300 further comprises a reinflatable bag or pouch.

17. The garment 200A of claim 15 wherein said deformable reservoir 300 further comprises an inlet 320, said inlet 320 allowing fluid to enter said deformable reservoir 300 therethrough.

18. The garment 200A of claim 17 wherein said inlet 320 further comprises a one-way valve, said one-way valve allowing said fluid to enter and not exit said deformable reservoir 300.

19. The garment 200A of claim 15 wherein said outlet 310 of said deformable reservoir 300 comprises a one-way valve, said one-way valve allowing said fluid to exit and not enter said deformable reservoir 300.

20. The garment 200A of claim 15 wherein said application of a pressure to said reservoir operatively conveys said fluid from said deformable reservoir 300, through said outlet 310 and into said duct 400, through said nozzle 410 and onto said upper torso, chest and neck area 520 of said wearer.
